(19) United States
(12) Patent Application Publication
(10) Pub. No.: US 2017/0189429 A1
(43) Pub. Date: Jul. 6, 2017

(54) Treating Arrhythmia with Mitochondrial-Targeted Antioxidants

(71) Applicant: Rhode Island Hospital, Providence, RI (US)

(72) Inventor: Samuel Dudley, Providence, RI (US)

(21) Appl. No.: 15/325,230

(22) PCT Filed: Jul. 10, 2015
(86) PCT No.: PCT/US15/40046

§ 371 Date: Jan. 10, 2017

Related U.S. Application Data
(60) Provisional application No. 62/022,986, filed on Jul. 10, 2014.

(51) Int. Cl. A61K 31/675; A61K 31/66; A61K 31/7084; A61L 31/16; A61L 29/16

(52) U.S. Cl. CPC A61K 31/675; A61L 31/16; A61L 29/16; A61K 31/7084; A61K 31/66; A61L 2300/40

(57) ABSTRACT

This invention relates to reducing the risk of arrhythmias as a result of an acute cardiovascular disorder or injury such as a myocardial infarction by using mitochondrial-targeted antioxidants to improve cardiac function.

### CROSS-REFERENCE TO RELATED APPLICATION

This application which claims priority to U.S. Provisional Application No. 62/022,986, filed on Jul. 10, 2014, the contents of all of which are incorporated herein by reference in its entirety.

### STATEMENT AS TO FEDERALLY SPONSORED RESEARCH

This invention was made with government support under 1R01HL106592-01A1, RO1 HL104025 (SCD), R41 HL112355 (SCD) awarded by National Institute of Health (NIH) and BX000859 (SCD), a Veterans Affairs MERIT grant. The government has certain rights in the invention.

### FIELD OF THE INVENTION

This invention relates to heart disease.

### BACKGROUND OF THE INVENTION

Patients at arrhythmic risk may suffer from a downregulation of cardiac sodium current, which causes a slowing of cardiac conduction and can result in sudden cardiac death (SCD). Current antiarrhythmic medications target one or several ion channels in the myocardium and inhibit their activity to restore proper cardiac current and output but can be associated with side effects and disadvantages. Therefore, there is a need to develop treatment options.

### SUMMARY OF THE INVENTION

The invention addresses this need and is based on a method of treating and/or ameliorating acute arrhythmia associated with an altered sodium channel current (I_{Na}) of a cardiac cell by administering a mitochondria-targeted antioxidant to a subject. The subject, e.g., human, is characterized as having an acute cardiac pathology, e.g., suffering from acute periods of myocardial ischemia or infarction and or cardiogenic shock. Such conditions include an increased risk of cardiac arrhythmias (for example atrial fibrillation or ventricle arrhythmias, including ventricular tachycardia and ventricular fibrillation). In some examples, the subject is characterized as suffering from acute myocardial infarction, acute myocarditis or acute cardiac arrest. The acute phase of a myocardial infarction, may manifest into recurrent episodes of acute cardiac electrical instability (for example, an electrical storm), characterized by sustained ventricular arrhythmia, within a 24 hour period. The ventricular arrhythmia includes ventricular tachycardia (VT), ventricular fibrillation (VF), or implantable cardioverter-defibrillator (ICD).

In some examples, the mitochondria-targeted antioxidant includes 2-(2,2,6,6-tetramethylpiperidin-1-oxyl-4-ylamino)-2-oxoethyl)triphenylphosphonium chloride (mitoTEMPO), 10-(6′-Ubiquinolyl)decylthriphenylphosphonium bromide (MitoQ) or, β-nicotinamide adenine dinucleotide.

The mitochondrial-targeted antioxidant is administered within the peri-infarction period of an acute myocardial infarction, and the peri-infarction period is within 48 hours of the index myocardial infraction, or a post-infarction period (for example, after 48 hours of said myocardial infarction). For example, the antioxidant is administered within 1, 1, 5, 30 minutes or within 1, 6, 12, 24, 36, or 48 hours of an acute cardiac event. The antioxidant may also be administered 2, 3, 5, 7, days or weeks after an acute event.

The subject may be a human patient or another mammal suffering from a disease that increases the risk of developing arrhythmias, e.g. past history of heart disease or having suffered a cardiac event such as a myocardial infarction, as well as other pathologies with cardiac implications such as diabetes mellitus, pathological cardiac hypertrophy, myocardial ischemia/reperfusion, ischemic cardiomyopathy, non-ischemic cardiomyopathy, heart failure, hypertension, atherosclerosis, valvular or coronary artery disease.

An arrhythmia is a disorder of the heart rate (pulse) or heart rhythm, such as beating too fast (tachycardia), too slow (bradycardia), or irregularly. Tests used to diagnose an arrhythmia or determine its cause include electrocardiogram, Holter monitor, transtelephonic monitor/event monitor, stress test, echocardiogram, cardiac catheterization, electrophysiology study (EPS), and/or head-up tilt table test. Mitochondrially-targeted antioxidants are useful as a therapeutic agent to elevate cardiac sodium channels, thereby treating, e.g., reducing arrhythmia after myocardial infarction.

In some examples, the mitochondrial-targeted small molecule compound includes a small molecule or lipo-tagged peptide. Small molecule compounds are less than 1000 daltons in molecular mass. Whether an organic compound or peptide, a small molecule mitochondria-targeted compound is between 50-1000 daltons, e.g., less than 750 daltons, 500 daltons, 250 daltons or 100 daltons, in molecular mass and preferentially accumulates in the mitochondria of a cell. Small molecules include pharmaceutically active organic agents, biological agents, or peptides.

A mitochondrial-targeted antioxidant or the pharmaceutically acceptable salt thereof is administered shortly after, e.g., within moments or hours, e.g., 6, 12, 24, 36, 48 hours or within at least 7 days after a subject has suffered or has been diagnosed with having suffered a myocardial infarction. The invention includes a mitochondrial-targeted antioxidant or the pharmaceutically acceptable salt thereof, which is administered minutes or up to 7 to 21 days after a subject has been diagnosed with having suffered a myocardial infarction. The invention includes a mitochondrial-targeted antioxidant or the pharmaceutically acceptable salt thereof, which is administered before, during, or after surgical treatment of acute arrhythmia of a subject. For example, a mitochondrial-targeted antioxidant or the pharmaceutically acceptable salt thereof, is administered is administered 24 hours to 1 minute before the surgical treatment of acute arrhythmia or 1 minute to 2 days (or 7 days, 21 days, or a month) after a surgical treatment of acute arrhythmia. In some examples, the acute arrhythmia is associated or coincident with myocardial infarction. In embodiments, the acute arrhythmia results in sudden cardiac arrest of the subject.

As used herein, an “isolated” or “purified” nucleic acid molecule, polynucleotide, polypeptide, protein or organic compound such as a small molecule (e.g., those described below), is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. Purified also defines a degree of sterility that is safe for administration to a human subject, e.g., lacking infectious or toxic agents.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

For example, the composition comprises the following mitochondrial-targeted antioxidants, which may be administered to a subject to treat and/or ameliorate arrhythmias associated with an altered sodium channel current: [2-(3,4-Dihydro-6-hydroxy-2,5,7,8-tetramethyl-2H-1-benzopyran-2-yl)ethyl]triphenylphosphonium bromide (MitoVit E), 10-(6′-Ubiquinolyl)decylthriphenylphosphonium bromide (MitoQ), [4-[4-[[(1,1-dimethylethyl)oxidoimino]methyl]phenoxy]butyl]triphenylphosphonium bromide (MitoPBN), 2-[4-(4-triphenylphosphoniobutoxy)phenyl]-1,2-benzoisoselenazol)-3(2H)-one iodide (MitoPeroxidase), 2-(2,2,6,6-tetramethylpiperidin-1-oxyl-4-ylamino)-2-oxoethyl)triphenylphosphonium chloride (MitoTEMPO), N-acetyl-L-cysteine choline ester (MitoGSH), glutathione choline ester (MitoGSH), SS-02 tetrapepetide (Dmt-D-Arg-Phe, Lys-NH₂), SS-20 tetrapepetide (Phe-D-Arg-Phe-Lys-NH₂), and SS-31 tetrapeptide (D-Arg-Dmt-Lys-Phe-NH₂).

In another aspect, a pharmaceutical composition of a mitochondrial-targeted antioxidant comprises a compound of formula:

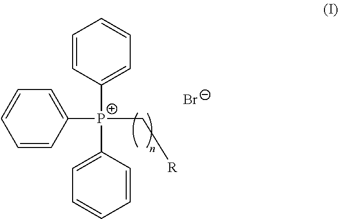

or pharmaceutically acceptable salt thereof
wherein
n is an integer from 0 to 15;
R is selected from a group consisting of CH₃,

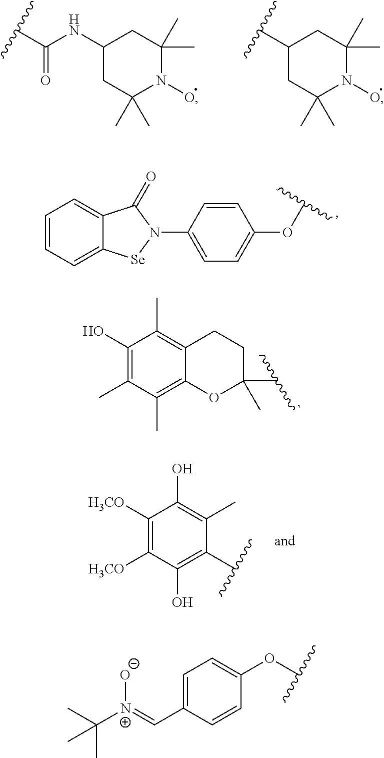

and a pharmaceutical acceptable carrier, diluent or excipient. These pharmaceutical compositions of mitochondrial-targeted antioxidants may also be used in suppressing or reducing mitochondrial reactive oxygen species (ROS) production in a cardiac cell as well as modulating or controlling sodium channel current of a cardiac cell.

A pharmaceutically effective amount of the antioxidant is administered to a subject experiencing acute arrhythmia in order to restore the sodium current (I_{Na}) to at least 80% of normal activity. The mechanism by which the sodium current (I_{Na}) is restored occurs via the reduction or suppression of mitochondrial reactive ROS production upon mitochondrial-targeted antioxidants administration. The antioxidant is administered at a dose in the range of 0.01-5.0 mg/kg once or twice a day, preferably 0.025 mg/kg once or twice a day. For example, patients receive one to three treatments of 40 mgs of antioxidant tablets or 80 mgs of antioxidant tablets such as MitoTEMP or MitoQ.

The antioxidant is administered into a lumen such as an artery or vein, or, subcutaneously, intraperitoneally , intravitreally, or orally. The subject to be treated is diagnosed as having suffered a myocardial infarction at least 24 hours prior to administering a mitochondrially-targeted antioxidant. For example, the therapeutic agent is administered at least 1, 2, 5, 7 days or more after a myocardial infarction, e.g., days after infarction, one, two, three or more weeks after infarction, or even months after infarction.

The subject can be treated with a combination of medications. For example, the classes of medications used in combination therapy may include other anti-arrhythmics, anti-clotting agents (anticoagulants), anti-hypertensives, cholesterol-lowering medications, inotropic and cardiotonic medications, pain relievers, and thrombolytic medical therapy.

Open surgical treatment for arrhythmias is usually done only when all other appropriate options, including minimally invasive surgical procedures, have failed. Surgical ablation is a major surgical procedure requiring general anesthesia. The chest is opened, exposing the heart. The site of the arrhythmia is located and the tissue is destroyed or removed in order to eliminate the source of the arrhythmia. This is typically done at the time of a concurrent cardiac surgical procedure such as cardiac bypass surgery or valve repair/replacement, and mitochondrial-targeted antioxidants are administered to the tissue in conjunction with the surgery.

In some examples, a patient undergoing cardiovascular surgery is administered a mitochondrial-targeted antioxidant or pharmaceutically acceptable salt thereof that is coated onto a surgical device comprising of a stent, an angioplasty balloon, drug eluting suture, staple or a tack.

Other features and advantages of the invention will be apparent from the following description of the preferred embodiments thereof, and from the claims. All references cited herein are hereby incorporated by reference.

### BRIEF DESCRIPTION OF THE DRAWINGS

FIG. 1A is a graph showing a series of sodium current traces measuring a I_{Na} reduction induced by NADH (nicotinamide adenine dinucleotide) (SCN5A+100 μM [NADH]) compared to normal I_{Na} (SCN5A), which can be restored by mitoTEMPO (SCN5A+100 μM [NADH]+5 μM [mitoTEMPO]). SCN5A: sodium channel, voltage gated type V alpha subunit

FIG. 1B is a bar graph showing I_{Na} reduction induced by NADH (SCN5A+100 μM [NADH]) compared to normal I_{Na} (SCN5A), which can be restored by mitoTEMPO (SCN5A+100 μM [NADH]+5 μM [mitoTEMPO]), which is also compared to mitoTEMPO (5 μM [mitoTEMPO]).

FIG. 2 is a bar graph showing Naᵥ 1.5 protein expression of isolated LV (left ventricle) cardiomyoctes from the anterior and lateral wall of Sham, MI (Myocardial Infarction) and MI mice treated with mitoTEMPO (MI-mitoTEMPO).

FIG. 3A is a blot showing Naᵥ 1.5 channel protein level expressions and GAPDH (Glyceraldehyde 3-phosphate dehydrogenase) protein level expressions in heart tissue of Sham, MI mice and MI mice treated with mitoTEMPO (MI tx MT).

FIG. 3B is a bar graph showing Naᵥ1.5/GAPDH protein expression in Sham, MI mice and MI mice treated with mitoTEMPO (MI tx MT).

FIG. 4A is a graphic showing a series of flow cytometry graphics for measuring mitoROS levels in cardiomyocytes of background (no stain), Sham, MI mice and MI mice treated with mitoTEMPO (MI tx MT).

FIG. 4B is a bar graph showing measured ROS levels of cardiomyocytes from Sham, MI mice and MI mice treated with mitoTEMPO (MI tx MT) co-stained with MitoSOX Red.

FIG. 4C is a bar graph showing measured ROS levels of cardiomyocytes from Sham, MI mice and MI mice treated with mitoTEMPO (MI tx MT) co-stained with MitoTracker Green.

FIG. 5A is a blot showing Connexin 43 protein level expressions and Glyceraldehyde 3-phosphate dehydrogenase (GAPDH) protein level expressions in heart tissue of Sham, MI mice and MI mice treated with mitoTEMPO (MI tx MT).

FIG. 5B is a bar graph showing Connexin 43/GAPDH protein expression in Sham, MI mice and MI mice treated with mitoTEMPO (MI tx MT).

FIG. 6A is a photo showing representative confocal microscopy images of myocytes of 3 groups. Scale bar, 10 μm. Mitochondrial ROS production in response to intracellular NADH was monitored by MitoSox Red with SCNSA cells and myocytes. The control groups were untreated, the pyruvate and lactate (PL) groups were treated with 1 and 10 mmol/L PL buffer, respectively, for 10 minutes, and the NAD-PL groups were incubated with 500 μmol/L NAD⁺ for ˜6 hours and then treated with PL buffer for 10 minutes.

FIG. 6B is a bar graph showing relative MitoSox Red fluorescent intensity.

***P<0.001 vs the untreated cells or NAD-PL groups. For each group, 9 to 16 samples were averaged.

FIG. 6C is a photo showing that mitochondrial ROS levels were increased in DOCA (deoxycorticosterone) myopathic mice and reduced by NAD treatment (500 μmol/L). Representative confocal microscopy images of mitochondrial ROS levels were obtained with treatment of myocytes monitored with MitoTracker Green and MitoSox Red. The white scale bar is 20 μm. The extremely red cells are dying myocytes that have very high levels of ROS.

FIG. 7A is a bar graph showing that extracellular NAD reversed the inhibition of NADH on cardiac I_{Na} in a dose-dependent manner. The SCNSA group is the untreated cells group, and the others were treated with 100 μmol/L NADH in the absence or presence of NAD from 50 to 1000 μmol/L.

FIG. 7B is a bar graph showing that the peak I_{Na} of DOCA mouse cardiomyocytes was significantly decreased compared with the sham group. Treatment of NAD⁺ at 500 μmol/L to DOCA myocytes restored I_{Na} to the sham level. NAD⁺ restored I_{Na} decreased by mitoROS in PL-treated cells and in DOCA myocytes.

FIG. 8A is a bar graph showing that extracellular NAD reversed the inhibition of NADH on cardiac I_{Na} in a dose-dependent manner. The SCN5A group is the untreated cells group, and the others were treated with 100 μmol/L NADH in the absence or presence of NAD⁺ from 50 to 1000 μmol/L.

FIG. 8B is a bar graph showing that the peak I_{Na} of DOCA mouse cardiomyocytes was significantly decreased compared with the sham group. Treatment of NAD⁺ at 500 μmol/L to DOCA myocytes restored I_{Na} to the sham level.

FIG. 9A is a bar graph showing that MitoQ restored I_{Na} that was decreased by antimycin A. The peak I_{Na} at −30 mV in adult mouse cardiomyocytes was decreased in antimycin A (5-10 μM) compared with SCNSA alone. Treatment using MitoQ at 10 nM with a 1-2 hour preincubation time restored I_{Na}. The SCNSA group is the untreated group. For patch clamp, MitoQ and antimycin A were also applied to the patch clamp bath solution during the entire time of patching, respectively or together. I_{Na} was measured at room temperature. Data shown in FIGS. 9A and 9B were obtained from 3 different mice, and 16-28 cells were used on average in each group.

FIG. 9B is a bar graph showing that Mito Q decreased ROS that was generated by antimycin A. ROS density in adult mouse cardiomyocytes was measured using MitoSox dye. Adult mouse cardiomyocytes were isolated, and MitoQ was applied for 1-2 hours pretreatment.

### DETAILED DESCRIPTION

Cardiac arrhythmias and conduction disturbances are commonly observed in patients with myocardial infarction. Arrhythmias are acutely treated either with medications or with electrotherapy. Electrotherapy includes any of the following types: external defibrillation or R-wave-synchronized cardioversion; antitachycardic stimulation, e.g., to terminate ventricular tachycardias or atrial flutter; antibradycardic stimulation, which, in the acute setting, usually consists of temporary transvenous or transcutaneous pacemaker treatment.

Bradyarrhythmia is characterized by an effective ventricular rate is less than 60 beats per minute. Tachycardia is defined as a heart rate above 100 beats per minute, though a symptomatic or hemodynamically relevant emergency usually arises only when the heart rate is 150 beats per minute or higher. The mode of treatment in the acute situation depends on whether the patient is hemodynamically stable or unstable.

Hemodynamically unstable tachycardia with shock, alteration of consciousness, or pulmonary edema is treated as soon as possible with cardioversion and/or defibrillation and/or medication. Hemodynamically unstable tachycardia with a narrow QRS (Q, R and S waves of graphical deflections on an electrocardiogram (EKG or ECG)) complex, e.g., due to atrial fibrillation or flutter, usually responds to low defibrillation energies of 50 or 100 joules. On the other hand, polymorphic ventricular tachycardia or ventricular fibrillation is treated primarily with defibrillation energies of at least 200 to 300 joules.

Types of Acute Cardiac Arrhythmic Conditions

Acute arrhythmia is caused by abnormal heart rhythms called arrhythmias, and can lead to sudden death. The most common life-threatening arrhythmia is ventricular fibrillation, which is an erratic, disorganized firing of impulses from the ventricles (the heart's lower chambers). When this occurs, the heart is unable to pump blood and death occurs within minutes, if left untreated. SCD is a sudden, unexpected death caused by loss of heart function (sudden cardiac arrest).

Acute myocardial infarction (AMI): AMI remains the leading cause of mortality worldwide and occurs when myocardial ischemia (a diminished blood supply to the heart), exceeds a critical threshold and overwhelms myocardial cellular repair mechanisms designed to maintain normal operating function and homeostasis. Acute MI is accompanied by significant intracellular and extracellular ionic and metabolic alterations. Arrhythmia may occur shortly after an infarction and the risk increases with a large infarct, low overall heart function, low contractile function, increased cardiac ectopy, and or VT.

The peri-infarction period, generally accepted as the time within 48 hours of the index myocardial infarction, is a period when arrhythmias are most likely to be seen. Arrhythmias developing in the post-infarction period (after 48 hours), have been demonstrated to be associated with an adverse outcome. In the post-infarction period, impaired vagal tone, as documented by decreased baroreflex sensitivity and heart rate variability, has been associated with increased inducibility of sustained monomorphic ventricular tachycardia and with sudden death.

Myocarditis: Myocarditis is clinically and pathologically defined as the inflammation of the myocardium. Myocarditis is frequently caused by infection by common viruses and is often an autoimmune reaction. During and after the infection, the immune system attacks cardiac myosin which may lead to ventricular arrhythmias and high-degree heart block.

Acute cardiac arrest: Acute cardiac arrest occurs suddenly and often without warning. It is triggered by an electrical malfunction in the heart that causes an irregular heartbeat, and the heart beats dangerously fast. The ventricles may flutter or quiver (ventricular fibrillation), and blood is not delivered to the body. The most common arrhythmia caused by acute cardiac arrest is ventricular fibrillation, when rapid and erratic electrical impulses cause the ventricles to quiver instead of pump blood. In the first few minutes, the greatest concern is that blood flow to the brain will be reduced so drastically that a person will lose consciousness. Death follows unless emergency treatment is begun immediately.

Atrial fibrillation (AF): Atrial fibrillation is the most common type of serious arrhythmia and involves a very fast and irregular contraction of the atria. In AF, the heart's electrical signals do not begin in the sinoatrial (SA) node (located in the right atrium), but rather begin in another part of the atria or in nearby pulmonary veins. This event causes signals to spread throughout the atria in a rapid and disorganized way and causes the walls of the atria to quiver very fast (fibrillate) instead of beating normally. As a result, the atria fail to pump blood into the ventricles. Two major complications of AF include stroke and heart failure. If blood pools in the atria, blood clots can form which can then cause a stroke. AF can also lead to heart failure, because the ventricles beat very quickly and cannot completely fill with blood, leading to an inability to pump enough blood to the lungs and body. Damage to the electrical system causes AF, which is most often the result of other conditions such as high blood pressure, coronary heart disease and rheumatic heart disease. An overactive thyroid gland, heavy alcohol use and increased age and may also lead to AF.

Atrial flutter: Atrial flutter is similar to AF, where the heart's electrical signals spread through the atria in a fast and regular (instead of irregular) rhythm. Atrial flutter is usually more organized and regular than atrial fibrillation. This arrhythmia occurs most often in people with heart disease and in the first week after heart surgery. It often converts to atrial fibrillation.

Ventricular arrhythmias: Ventricular arrhythmias can be dangerous and usually require medical treatment immediately. The arrhythmias start in the heart's lower chambers and the ventricles. Two types of ventricular arrhythmia include ventricular tachycardia and ventricular fibrillation (VF)

Ventricular tachycardia (VT): VT is characterized by a rapid heart rhythm originating from the lower chambers (or ventricles) of the heart. The rapid rate prevents the heart from filling adequately with blood; therefore, less blood is able to pump through the body.

An electrical storm or (VT-storm) is an increasingly common and life-threatening syndrome that involves recurrent episodes of ventricular arrhythmias. It is defined as 3 or more sustained episodes of ventricular tachycardia (VT), ventricular fibrillation (VF) or appropriate implantable cardioverter-defibrillator (ICD) shocks during a 24-hour period. A sustained VT storm lasts 30 seconds, involves hemodynamic compromise, and often requires intervention to terminate the episode. Management of the electrical storm is challenging and requires a tailed approach to the underlying cause. The condition itself is manifested during the acute phase of a myocardial infarction (MI), a structural heart disease, or an arrhythmic syndrome. An important step in evaluating the condition is to identify and reverse the causative factors.

Ventricular fibrillation (VF): VF occurs if disorganized electrical signals make the ventricles quiver instead of pump normally. Without the ventricles pumping blood to the body, sudden cardiac arrest and death can occur within a few minutes. This is a medical emergency that must be treated with cardiopulmonary resuscitation (CPR) and defibrillation (an electrical shock to the heart) as soon as possible. VF can occur during or after a heart attack or in someone whose heart is already weak due to another condition. Torsades de pointes (torsades) is a type of VF that causes a unique pattern on an EKG test.

Sudden arrhythmic death syndrome (SADS): Sudden arrhythmic death syndrome (SADS) describes the sudden death due to cardiac arrest brought on by an arrhythmia, and genetic heart conditions. The most common cause of sudden death in the US is coronary artery disease, specifically because of poor oxygenation of the heart muscle. Several different types of ion channelpathies that cause life-threatening arrhythmias include: Long QT Syndrome (LQTS), Brugada Syndrome, CPVT (catecholaminergic polymorphic ventricular tachycardia), PCCD (progressive cardiac conduction defect), IVF (idiopathic ventricular fibrillation) and Sodium channel disease.

Long QT syndrome: The QT interval is the area on the electrocardiogram that represents the time it takes for the heart muscle to contract and then recover, or for the electrical impulse to fire impulses and then recharge. When the QT interval is longer than normal, it increases the risk for “torsade de pointes,” a life-threatening form of ventricular tachycardia. Long QT syndrome is an inherited condition that can cause sudden death in young people. It can be treated with antiarrhythmic drugs, pacemaker, electrical cardioversion, defibrillation, implanted cardioverter/defibrillator, or ablation therapy.

Brugada syndrome: Brugada syndrome is a condition in which the sodium channel behaves abnormally, in that the movement of sodium ions into the cells is restricted which results in changes in the ECG (electrocardiogram), with no structural abnormalities. Without appropriate treatment, the outlook for patients can be poor, and treatment for patients with an abnormal ECG, and who are asymptomatic is often very difficult.

CPVT: CPVT is a rare condition found in young people and children and causes a particular type of arrhythmia. Two calcium ion channels are associated with CPVT, which regulate the release of calcium ions into the rest of the cell. If the channels do not function normally, the level of calcium inside the cell becomes too high, resulting in arrhythmia.

PCCD: PCCD is a rare condition where the heart's electrical impulses are conducted very slowly which results in the gradual development of “heart block” (the failure of the heart's electrical impulses to conduct properly from the top chambers to the bottom chambers). In some patients, PCCD is associated with sodium channel mutations that cause changes in channel behavior. Pacemakers, antiarrhytmic agents and ICDs (implantable cardioverter defibrillator) are used to treat/prevent the condition in patients.

IVF: IVF describes conditions responsible for life-threatening, fast heart rhythm disturbances in people who do not have any signs of heart disease.

### Antioxidant Therapy

The invention features mitochondrially targeted antioxidants as a therapy to raise cardiac sodium channels and treat acute arrhythmia after myocardial infarction. Ischemic cardiomyopathy leads to downregulation of cardiac Naᵥ1.5 current and overproduction of mitochondrial ROS, e.g., a patient to be treated is identified as having such a downregulation or infarction-associated reduction in cardiac Naᵥ1.5 current. Compounds belonging to the class of mitochondria-targeted antioxidants mitigate these changes and reduce arrhythmic risk after myocardial infarction. The antioxidants are administered in an amount and formulation that leads to an increase in peak sodium current level of at least 5%, 10%, 20%, 50%, 75%, 90%, 2-fold or more compared to the level in the absence of administration of the therapeutic agent. For example, the sodium level in a target patient (whose levels have been pathologically reduced as a result of myocardial infarction) are restored to at least 80%, 90%, 95%, and even back to 100% of normal following treatment, thereby conferring a clinical benefit.

Cardiac ion channel homoeostasis and structural remodeling are associated with elevated ROS and oxidative stress. Oxidative stress occurs when ROS such as free radicals react with and damage biological molecules, cells and tissues, a major contributing factor underlying a wide range of diseases and pathologies such as cardiac arrhythmia. Pathologies include Alzheimer's disease, Parkinson's disease, fatty liver disease, diabetes mellitus, pathological cardiac hypertrophy, myocardial ischemia/reperfusion, ischemic cardiomyopathy, heart failure, hypertension, atherosclerosis, valvular disease, coronary artery disease, and cardiotoxicity in organ preservation or transplantation.

The data described herein indicate that the risk of arrhythmia due to a cardiovascular disorder or injury such as a myocardial infarction is reduced by mitochondrial-targeted antioxidants to improve cardiac function. Exemplary mitochondrial-targeted antioxidants are described above as well as in U.S. Patent Pub Nos. 20120288486 and 20120308542, each of which are hereby incorporated by reference Others are described in the art, e.g., U.S. Pat. No. 6,331,532, hereby incorporated by reference and Jin et al. Biochim Biophys Acta. 2013 Sep. 20: S0925-4439, Szeto et al. Antioxidants & Redox Signaling, 2008, 10 (3), 601-618, Sheu et al. Biochim. Biophys. Acta 2006, 1762, 256-365, Li et al. J. Hematology & Oncology 2013, 6, 1-19 (e.g, p. 10, Table 2), Smith et al. Cell 2013, 33, 341-352, Smith et al., Trends in Pharm. Sci., 2012, 33, 341-552 (e.g., p. 347, Table 1) and Murphy et al. Annu. Rev. Pharmacol. Toxicol. 2007, 47, 629-656 (e.g., pp 635-645, antioxidants conjugated to a triphenylphosphonium (TPP) moiety; e.g., p. 646, exemplary oral dosing at 80 mg (1 mg/kg) of TPP compositions). In addition conjugation to a TPP cation, triphenylarsine, triphenylphosphine, triphenyl amine, or benzylammonium cations can be used to make mitochondrially-targeted therapeutic agents. All of the foregoing references are hereby incorporated by reference.

Unlike conventional antioxidants, therapeutic efforts using conventional antioxidants such as vitamin A, Vitamin E, Ebselen, and Ubiquinone-10 have generally been disappointing. To target ischemic cardiomyopathy, the methods described herein employ mitochondria-targeted antioxidants, which preferentially gain access to the mitochondria based on their lipophilicity and charge, which facilitates their passage directly through biological membranes and into the mitochondria. Due to their design to specifically accumulate within the mitochondria, they protect against cellular oxidative damage. One group of mitochondria-targeted antioxidants consists of a conventional antioxidant moiety, which is conjugated to a lipophilic cation such as triphenylphosphonium. TPP is effective at delivering conventional antioxidants to the mitochondria because these substrates are able to pass directly through phospholipid bilayers without requiring a specific uptake mechanism. Due to their high membrane potential TPP-modified antioxidants accumulate substantially within the mitochondria. Examples of TPP-modified antioxidants include [2-(3,4-Dihydro-6-hydroxy-2,5,7,8-tetramethyl-2H-1-benzopyran-2-yl)ethyl]triphenylphosphonium bromide (MitoVit E), 10-(6′-Ubiquinolyl)decyltriphenylphosphonium bromide (MitoQ), [4-[4-[[(1,1-dimethylethyl)oxidoimino]methyl]phenoxy]butyl]triphenylphosphonium bromide (MitoPBN), 2-[4-(4-triphenylphosphoniobutoxy)phenyl]-1,2-benzoisoselenazol)-3(2H)-one iodide (MitoPeroxidase), 2-(2,2,6,6-tetramethylpiperidin-1-oxyl-4-ylamino)-2-oxoethyl)triphenylphosphonium chloride (MitoTEMPO), N-acetyl-L-cysteine choline ester (MitoGSH), glutathione choline ester (MitoGSH), as well as β-nicotinamide adenine dinucleotide. Several of these agents have been studied in humans. For example, MitoQ has been evaluated in human studies of cardiac ischemia-reperfusion injuries and hypertension, mitoTEMPO has been studied to combat hypertension and mitoSNO has been studied in cardiac ischemia-reperfusion injury studies. Another class of compounds is the Szeto-Schiller peptides in particular SS-02 (Dmt-D-Arg-Phe-Lys-NH₂), SS-31 (D-Arg-Lys-Phe-NH₂) and SS-20 (Phe-D-Arg-Phe-Lys-NH₂), which are cell-permeable small peptides that selectively partition to the inner mitochondrial membrane. Common to all of these peptides is an alternating aromatic-cationic motif, with basic amino acid residues providing two positive charges. The free amine of the N-terminus of these peptides provides a third positive charge because the C-terminus has been amidated. Furthermore, these positive-charged peptides are not delivered into the mitochondrial matrix and therefore not limited to mitochondria with normal potential, which makes them attractive in studies of defective/damaged mitochondria. SS-31 has been investigated in cardiac ischemia-reperfusion injury studies in humans as well. All of these compounds are between 50-1000 daltons in molecular mass.

### Antioxidant Treatment Concurrent With Acute Cardiac Procedures

A blocked blood vessel is treated with a variety of surgical or minimally invasive procedures. One option is a percutaneous coronary intervention (PCI), such as balloon angioplasty. Cardiologists perform an angioplasty, which opens narrowed arteries. Using a catheter that has a small balloon on its tip, the balloon is inflated at the blockage site in the artery to flatten or compress the plaque against the artery wall.

Another surgical widening option is a stent, a small, mesh-like device made of metal (such as stainless steel or cobalt). When a stent is placed inside of a coronary artery, it acts as a support or scaffold, keeping the vessel open. By keeping the vessel open, the stent helps to improve blood flow to the heart muscle and reduce the pain of angina. The most common use for coronary stents is in the coronary arteries, into which a bare-metal stent, a drug-eluting stent, a bioabsorbable stent, a dual-therapy stent (combination of both drug and bioengineered stent), or occasionally a covered stent is inserted Like in an angioplasty procedure, a stent mounted onto a tiny balloon is opened inside of an artery to restore blood flow.

Alternatively, drug-eluting sutures, staples and tacks can be introduced into patient tissue undergoing cardiac bypass surgery.

The compounds described herein are coated onto or incorporated into the surgical tools, devices, or implants described above. Such structural elements may also include medications, e.g., anti-proliferative compounds or clotting inhibitors to reduce restenosis.

### Antioxidants for Cardiac Therapy

Mitochondria-targeted antioxidants are taken up into mitochondria following oral administration. However, additional modes of administration include intravenous, subcutaneous, intraperitoneal and intravitreal, as well as direct injection into heart muscle or infusion into cardiac blood vessels. The data indicate that treatment of patients with mitochondrial-targeted compounds leads to a clinical benefit.

Mitochrondrial antioxidants may be classified as follows:

Triphenylphosphonium-Based, Mitochondrially Targeted Antioxidants

This class includes MitoVit E, [2-(3,4-dihydro-6-hydroxy-2,5,7,8-tetramethyl-2H-1-benzopyran-2-yl)ethyl]triphenylphosphonium bromide; MitoQ, a mixture of 10-(6V-ubiquinolyl)decyltriphenylphosphonium bromide and 10-(6V-ubiquinonyl)decyltriphenylphosphonium Bromide; MitoPBN, [4-[4-[[(1,1-dimethylethyl)oxidoimino]-methyl]phenoxy]butyl]-triphenylphosphonium bromide. Antipodean Pharmaceuticals Inc.'s (www.antipodeanpharma.com) MitoQ has been tested in human clinical trials for non-cardiac indications.

Also within this class is MitoTEMPO, a mitochondrially targeted antioxidant and a specific scavenger of mitochondrial superoxide. MitoTEMPO is a combination of the antioxidant piperidine nitroxide TEMPO with the lipophilic cation triphenylphosphonium, giving MitoTEMPO the ability to pass through lipid bilayers with ease and accumulate several hundred-fold in mitochondria. MitoTEMPO has the formula (2-(2,2,6,6-Tetramethylpiperidin-1-oxyl-4-ylamino)-2-oxoethyl)triphenylphosphonium chloride monohydrate; C₂₉H₃₅N₂O₂ClP.H₂O; and a structure depicted below.

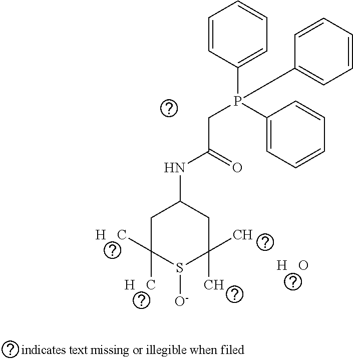

### Amino Acid- and Peptide-Based, Mitochondrially Targeted Antioxidants

This class of agents includes SS tetrapeptides. Examples include antioxidant SS tetrapeptides. SS-02, Dmt-D-Arg-Phe-Lys-NH2; SS-20, Phe-D-Arg-Phe-Lys-NH2; SS-31, D-Arg-Dmt-Lys-Phe-NH2; where “Dmt” is the antioxidant 2′,6′-dimethyltyrosine.

### Choline Esters of Glutathione and N-acetyl-1-cysteine

Examples of members of this class include MitoGSH, choline (glycinate) ester of glutathione; MitoNAC, choline ester of N-acetyl-1-cysteine.

Another class of mitochondria-targeted antioxidants includes 10(6′-plastoquinonyl) decylrhodamine 19 (SkQR1).

Such antioxidants are generally characterized as having a molecular mass of 50-100 daltons, e.g., MitoQ, 678.81 daltons; Mito TEMPO, approximately 510 daltons, SS-31, approximately 750 daltons.

The mitochondrially-targeted antioxidants described herein are utilized in therapeutic interventions to raise cardiac sodium channels and treat arrhythmia after myocardial infarction. In preferred embodiments, the therapeutic agents are not administered to patients whose primary diagnosis is cancer for treatment of the cancer or to reduce the toxicity of chemotherapeutic agents. For example, the antioxidants are preferably not administered in combination with a chemotherapeutic agent such as an anthracycline, e.g., doxorubicin.

### Downregulation of Cardiac Na+ Channel in Myocardial Infarction is Prevented by a Mitochondria-Targeted Antioxidant

The therapeutic administration of mitochondrial-targeted antioxidants after ischemic injury is used for the treatment and/or amelioration of arrhythmia. The effect of ischemia on cardiac Na₊channel (Naᵥ1.5) was studied in a rodent, e.g., mouse, model of myocardial infarction (MI). Treatment options were evaluated. Results of the studies demonstrated a statistically significant increase in I_{Na} activity as well as a statistically significant decrease of mitochondrial ROS when rodents where treated with a mitochondria-targeted antioxidant such as mitoTEMPO (0.7 mg/kg/day, intraperitoneally) after ischemic insult. The results indicated that ischemic cardiomyopathy leads to downregulation of cardiac Naᵥ1.5 currents and overproduction of mitochondrial ROS and that administration of mitochondria-targeted antioxidant mitigates these changes and reduces arrhythmic risk after myocardial infarction.

### EXAMPLES

### Downregulation of Cardiac Na⁺ Channel in Myocardial Infarction is Prevented by a Mitochondria-Targeted Antioxidant

The following materials and methods were used to generate the data described herein. MI was induced in 12-week old C57BL/6 mice by coronary artery occlusion. Sham-operated mice were used as controls. Two weeks following surgery, MI mice were either given a mitochondria-targeted antioxidant, mitoTEMPO (0.7 mg/kg/day, intraperitoneally), or left untreated for two weeks. Cardiomyocytes isolated from the scar border of MI mice or from the left ventricular (LV) anterior wall of sham-operated mice were utilized for whole-cell patch clamp recording of Na⁺currents (I_{Na}) and for measurements of mitochondrial reactive oxygen species (mitoROS) using flow cytometry. Naᵥ1.5 protein expression levels were determined in the LV from MI and sham-operated mice. Echocardiography was performed 2- and 4-weeks following MI. Oxidized nicotinamide adenine dinculeotide used for experiments was purchased from Sigma-Aldrich, catalog number N1636 “β-Nicotinamide adenine dinucleotide hydrate, ≧99%, Synonyms: NAD⁺, β-DPN, β-NAD, Coenzyme 1, Cozymase, DPN, Diphosphopyridine nucleotide, NAD, Nadide” and dissolved in water. MitoQ was used at 10 nM.

### Example 1

### Ischemic Cardiomyopathy Leads to Down Regulation of Cardiac Naᵥ1.5 Currents and Overproduction of Mitochondrial ROS

The peak I_{Na} densities of the isolated LV cardiomyocytes were significantly lower (P<0.05) in MI (−14.3±1.4 pA/pF), compared to sham (−24.0±1.8 pA/pF). The mitoROS levels were elevated to 1.5±0.2 fold in MI mice (P<0.05). I_{Na} was increased (−19.4±0.8 pA/pF, P<0.05) and mitoROS was decreased to 1.2±0.2 fold (P<0.05) with mitoTEMPO treatment. The Naᵥ1.5 channel protein level was not altered in the heart tissue of MI mice. There were no significant differences in echocardiography parameters between untreated and mitoTEMPO groups to explain the increase in I_{Na}.

These results indicated that ischemic cardiomyopathy leads to down regulation of cardiac Naᵥ1.5 currents and overproduction of mitochondrial ROS and that mitochondria-targeted antioxidants are useful to reduce arrhythmic risk after myocardial infarction.

### Example 2

### Downregulation of Cardiac Na⁺ Channel is Prevented by Myocardial Dial-Targeted Antioxidant in Myocardial Infraction

Following MI, patients are at increased risk for ventricular tachyarrhythmia and SCD. MI induces mitochondrial dysfunction, oxidative stress, and slowed conduction velocity, which is determined by the function of gap junctions and Naᵥ 1.5 in MI (Peters et al. Circulation 1993; 88:864-875; Rutledge et al. J Am Coll Cardiol 2014; 63:928-934; Pu et al. Circ Res 1997; 81:110-119). Mitochondrial ROS (mitoROS) overproduction causes reduced cardiac Na⁺ channel (Naᵥ1.5) current (I_{Na}) (Liu et al. Circ Res 2010; 107:967-974). MI was associated with mitoROS overproduction and reduced I_{Na}, which is ameliorated by a mitochondria-targeted antioxidant mitoTEMPO. This improvement is associated with changes in conduction velocity and inducibility of arrhythmias in a MI mouse model.

MI was induced in 12-week old C57BL/6 mice by coronary artery occlusion. Sham-operated mice were used as controls. Two weeks after surgery, MI mice were either given a mitochondria-targeted antioxidant, mitoTEMPO (0.7 mg/kg/day, intraperitoneally), or left untreated for two weeks (Rutledge C A et al. J Am Coll Cardiol 2014; 63:928-934).

Cardiomyocytes isolated from the scar border of MI mice or from the left ventricular (LV) anterior wall of sham-operated mice were utilized for whole-cell patch clamp recording of Na⁺ currents (I_{Na}) and for measurements of mitoROS using flow cytometry.

Whole cell I_{Na} were measured using the whole-cell patch clamp technique in voltage-clamp mode at room temperature.

Naᵥ1.5 protein expression was determined in the LV from MI and sham-operated mice. The primary antibody (rabbit anti-SCNSA, Alomone Labs) was diluted 1:200. Horseradish peroxidase-conjugated goat anti-rabbit IgG secondary antibody (Cell Signaling Technology) was diluted 1:5000. Actin (Santa Cruz Biotechnology) was used as a loading control (Liu et al. J Mol Cel Cardiol 2013; 54:25-34).

A BD LSR II (BD Biosciences) flow cytometer was used for high-throughput evaluation of mitoROS, mitochondrial volume, and cell survival. Suspended cardiomyocytes from sham (n=3), MI (n=5), and mitoTEMPO-treated MI (n=5) were co-stained with 5 μM MitoSOX Red and 100 nM MitoTracker Green. Cells were gated for size (forward scatter and side scatter gating) to disregard cell debris. Separate suspensions were stained with propidium iodide (10 μg/mL) and evaluated at 617 nm to measure for cell integrity. Five thousand gated cells were recorded in each sample. BD FACSDiva software was used for data recording and analysis.

### MitoROS Downregulate Cardiac Naᵥ1.5

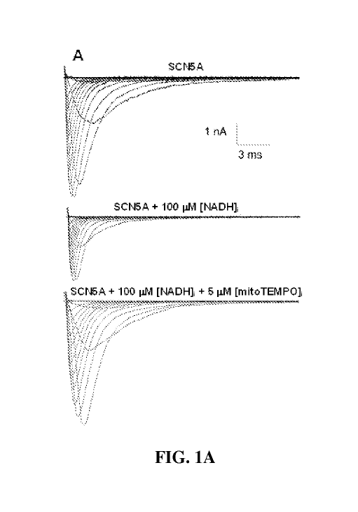

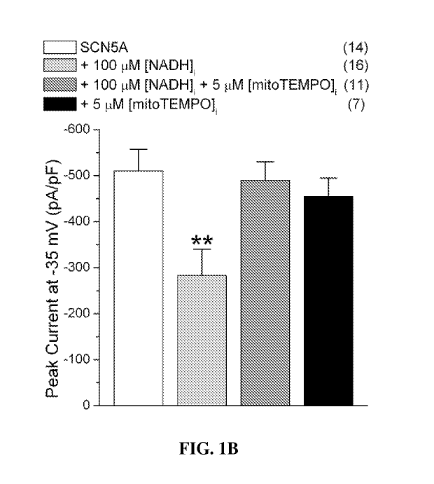

I_{Na} reduction induced by NADH is mediated by mitoROS, which can be restored by mitoTEMPO (FIG. 1A-B) (Liu et al. Circ Res 2010; 107:967-974).

### Cardiac Naᵥ1.5 Downregulation in MI

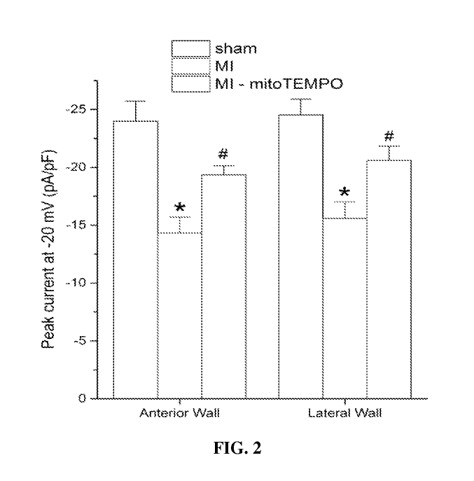

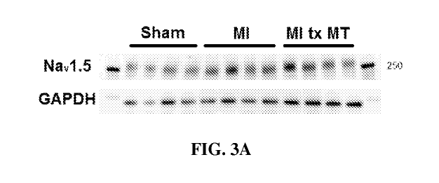

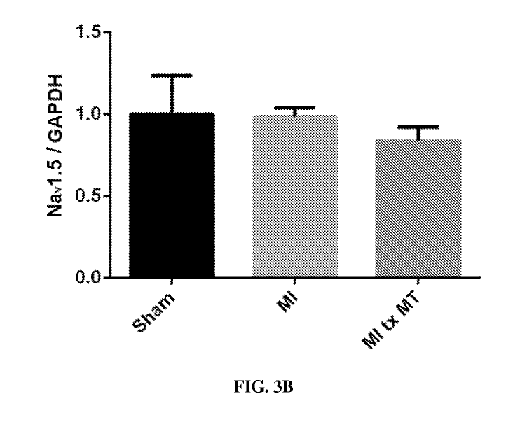

The peak I_{Na} densities of the isolated LV cardiomyocytes were significantly lower in MI mice (−14.3±1.4 pA/pF, *P<0.05 vs sham), compared to sham (−24.0±1.8 pA/pF). I_{Na} was recovered with mitoTEMPO treatment in MI mice (−19.4±0.8 pA/pF, #P<0.05 vs MI) (FIG. 2). The Naᵥ1.5 channel protein level was not altered in the heart tissue of MI mice (FIG. 3A-B).

### MitoROS Overproduction in MI

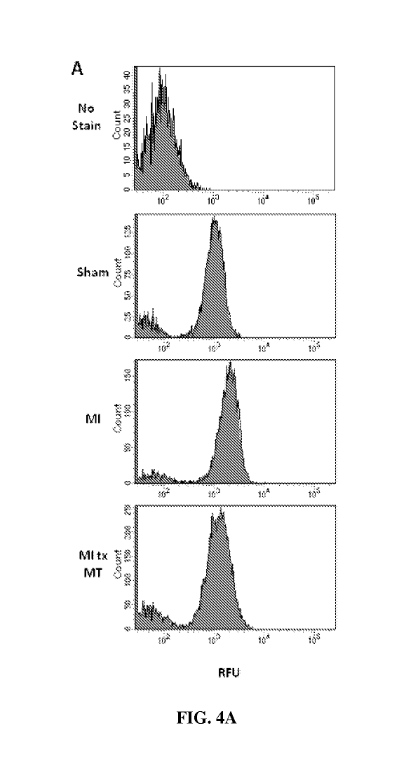

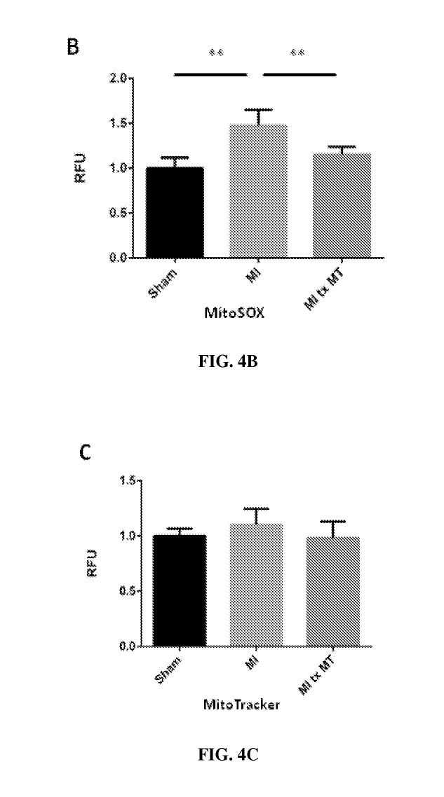

The mitoROS levels were elevated to 1.5±0.2 fold in cardiomyocytes of the scar border of MI mice heart tissue (P<0.05). MitoTEMPO treatment decreased the mitoROS level of MI mice back to the sham level (1.2±0.2 fold (P<0.05) (FIG. 4A-C).

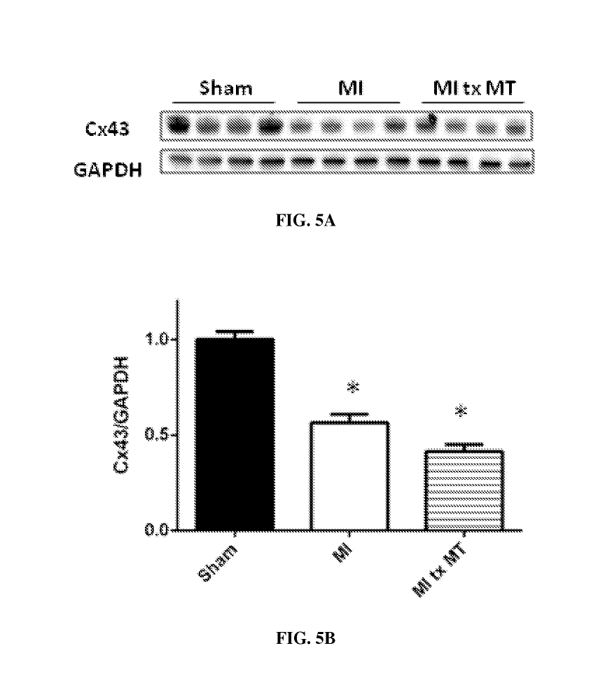

The mitoROS levels of cardiomyocytes of the lateral wall were not significantly affected in MI mice heart tissue (P>0.05). Connexin 43 levels were reduced to 56% of sham levels in MI mice (P<0.01) and 41% in mitoTEMPO-treated MI mice (P<0.01 compared to sham, p=ns compared to MI) (FIG. 5A-B).

These data indicate that ischemic cardiomyopathy leads to overproduction of mitochondrial ROS and down-regulation of cardiac Naᵥ1.5 currents. Mitochondria-targeted antioxidants, e.g., mitoTEMPO, mitigates these changes, indicating that mitochondrial ROS induce cardiac Naᵥ1.5 downregulation in MI. Downregulation of both connexin 43 and cardiac Naᵥ1.5 was found to contribute to the slow conduction velocity of MI tissue. MitoTEMPO showed no effect on connexin 43 downregulation in MI. Thus, decreasing mitochondrial ROS with mitochondria-targeted antioxidants such as mitoTEMPO is efficacious to reduce arrhythmic risk after myocardial infarction.

### Example 3

### NAD Decreased MitoROS Level and Increased by PL or in DOCA Myocytes

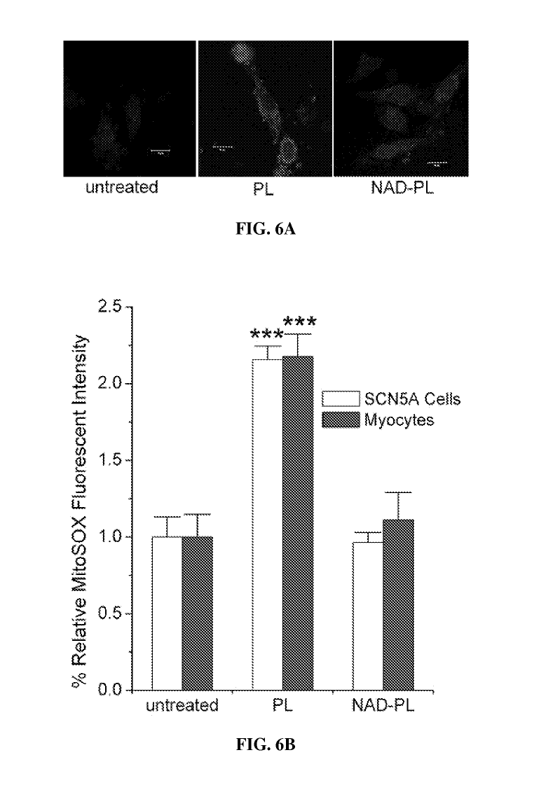

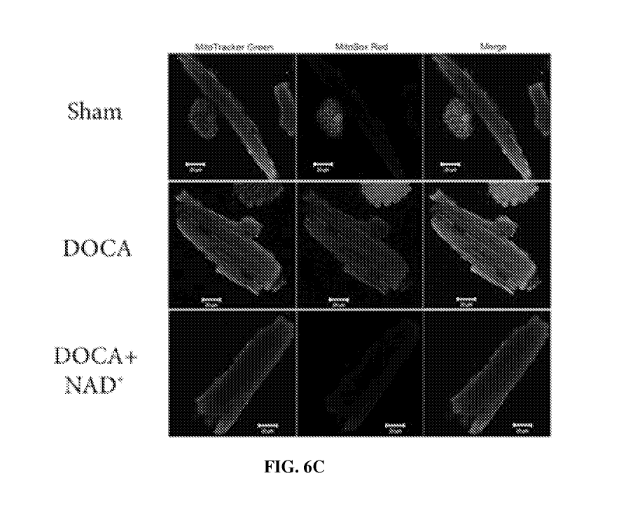

Mitochondrial ROS production in response to intracellular NADH was monitored by MitoSox Red with SCNSA cells and myocytes (FIGS. 6A and 6B). The control groups were untreated, the PL (pyruvate lactate) groups were treated with 1 and 10 mmol/L pyruvate and lactate (PL buffer), respectively, for 10 minutes, and the NAD-PL groups were incubated with 500 μmol/L NAD⁺ for ˜6 hours and then treated with PL buffer for 10 minutes. FIG. 6A shows representative confocal microscopy images of myocytes of 3 groups. Scale bar, 10 μm. Relative MitoSox Red fluorescent intensity. ***P<0.001 vs the untreated cells or NAD-PL groups is shown in FIG. 6B. For each group, 9 to 16 samples were averaged.

Mitochondrial ROS levels were increased in DOCA myopathic mice and reduced by NAD⁺ treatment (500 μmol/L) (FIG. 6C. Representative confocal microscopy images of mitochondrial ROS levels were obtained with treatment of myocytes monitored with MitoTracker Green and MitoSox Red. The white scale bar is 20 μm. The extremely red cells are dying myocytes that have very high levels of ROS.

### Example 4

### NAD⁺ Restored I_{Na} Decreased by MitoROS in PL-Treated Cells and in DOCA Myocytes

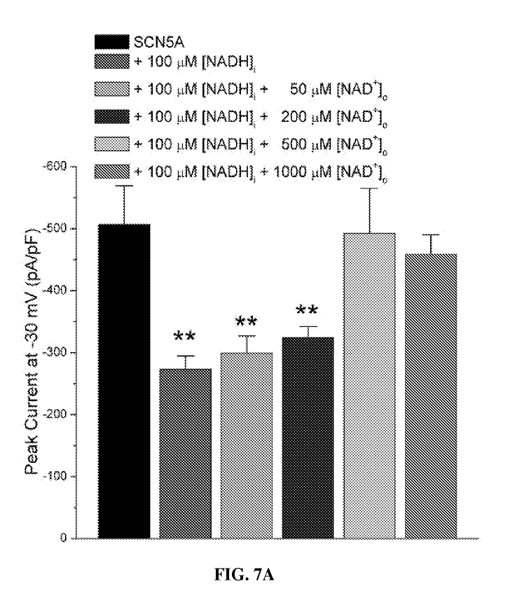

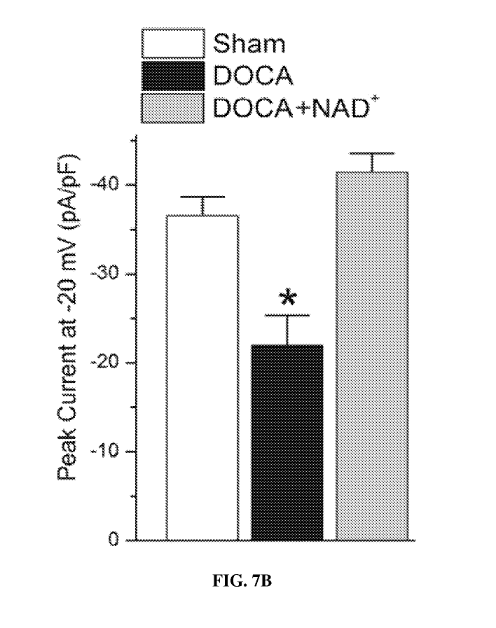

Extracellular NAD⁺ reversed the inhibition of NADH on cardiac I_{Na} in a dose-dependent manner (FIG. 7A). The SCN5A group is the untreated cells group, and the others were treated with 100 μmol/L NADH in the absence or presence of NAD from 50 to 1000 μmol/L.

The peak I_{Na} of DOCA mouse cardiomyocytes was significantly decreased compared with the sham group (FIG. 7B). Treatment of NAD at 500 μmol/L to DOCA myocytes restored I_{Na} to the sham level.

### Example 5

### In Vivo Animal Treatment Showed Decreased Mitochondrial ROS and Reduced I_{Na} in Cardiomyopathy With Injection of NAD

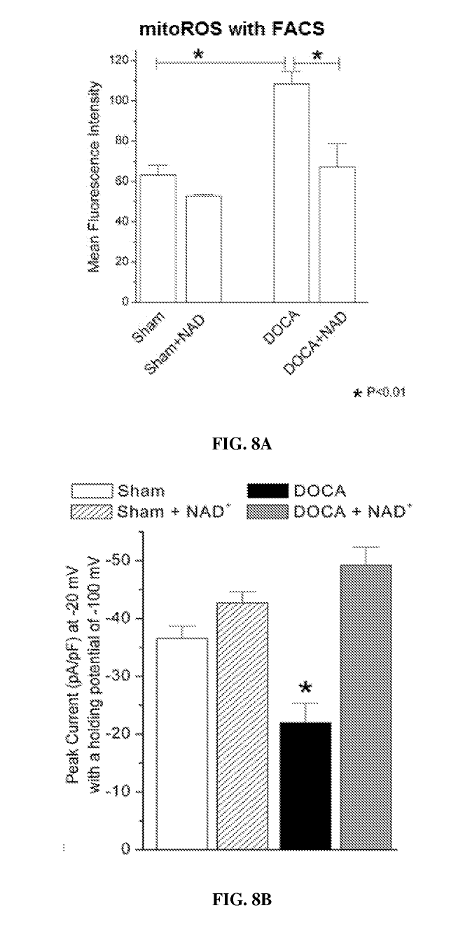

DOCA mice injected with NAD⁺ (100 mg/kg) in the neck showed decreased mitochondrial ROS (1.1±0.1-fold of sham, P>0.05). Three to four animals and ˜10,000 myocytes from each animal were tested in each group in flow cytometry (FIG. 8A).

Reduced I_{Na} in cardiomyopathy of DOCA was corrected by injection of NAD (100 mg/kg), which had no effect on the sham group (FIG. 8B).

### Example 6

### MitoQ Restored I_{Na} Decreased by Antimycin A in Adult Mouse Cardiomyocytes

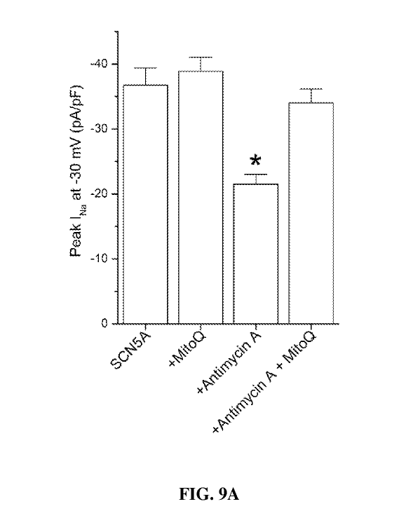

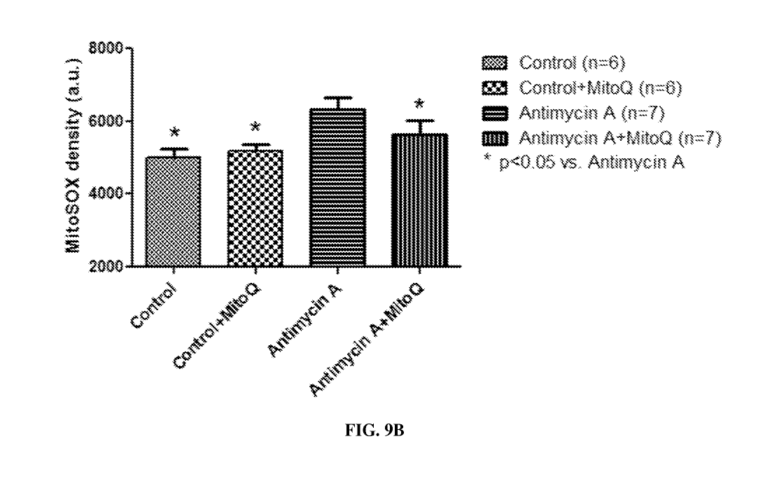

In addition to MitoTEMP and NAD⁺, another mitochondrial-targeted antioxidant, MitoQ, was tested. Antimycin C is an antibiotic that induces apoptosis and inhibits the mitochondrial electron transport chain, thereby inducing oxidative stress and generating reactive oxygen species. Because antimycin A is known to generate ROS (for example, increasing superoxide formation), it decreases the sodium channel current (I_{Na}). Additionally, antiomycin A causes damage to mitochondrial DNA, lipids, and proteins in cells treated with the antibiotic. MitoSOX Red is a fluorescent mitochondrial probe used to measure ROS production. Mitochondrial ROS production in response to increased antimycin A levels was monitored by MitoSox Red with SCNSA cells and myocytes (FIGS. 9A and 9B).

Isolated adult mouse cardiomyocytes were pretreated with MitoQ. The MitoQ groups were incubated with 10 nM MitoQ for 1-2 hours, and 5-15 μM antimycin A. FIG. 9A shows that treatment with MitoQ restored I_{Na}. FIG. 9B shows elevated MitoSOX density in antimycin A+MitoQ treated cardiomyocytes compared to control and control+MitoQ.

### Treatment of Arrhythmia With Mitochondrial-Targeted Antioxidants

The data described in these examples indicate that compounds in the class characterized as mitochondria-targeted antioxidants produce an antiarrhythmic effect. The effect is not compound specific demonstrated by three representative examples. Each of the three compounds tested, produce the effect and restore sodium channel voltages to clinically appropriate levels. These mitochondrial-targeted antioxidants are well-tolerated, safe, and orally active. Thus, the compounds are administered orally, sublingually or rectally as well are parenterally such as intravenously as well as local administration to affected tissue using coated devices or implantation of a drug delivery depot composition to treat arrhythmia associated with acute cardiac injury or dysfunction.

### OTHER EMBODIMENTS

While the invention has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the disclosure encompassed by the appended claims.

What is claimed:

1. A method of inhibiting acute cardiac arrhythmia, comprising administering a mitochondrial-targeted antioxidant, to a subject in need thereof.

2. The method of claim 1, wherein said mitochondrial-targeted antioxidant comprises 2-(2,2,6,6-tetramethylpiperidin-1-oxyl-4-ylamino)-2-oxoethyl)triphenylphosphonium chloride (mitoTEMPO), 10-(6′-Ubiquinolyl)decylthriphenylphosphonium bromide (MitoQ) or, β-nicotinamide adenine dinucleotide.

3. The method of claim 1, wherein said subject comprises acute myocardial infarction, acute myocarditis, acute cardiac arrest, or a ventricular tachycardia storm (VT-storm).

4. The method of claim 1 wherein said mitochondrial-targeted antioxidant is administered within the peri-infarct period.

5. The method of claim 1, wherein the mitochondrial-targeted antioxidant comprises [2-(3,4-Dihydro-6-hydroxy-2,5,7,8-tetramethyl-2H-1-benzopyran-2-yl)ethyl]triphenylphosphonium bromide (MitoVit E), 10-(6′-Ubiquinolyl)decylthriphenylphosphonium bromide (MitoQ), [4-[4-[[(1,1-dimethylethyl)oxidoimino]methyl]phenoxy]butyl]triphenylphosphonium bromide (MitoPBN), 2-[4-(4-triphenylphosphoniobutoxy)phenyl]-1,2-benzoisoselenazol)-3(2H)-one iodide (MitoPeroxidase), 2-(2,2,6,6-tetramethylpiperidin-1-oxyl-4-ylamino)-2-oxoethyl)triphenylphosphonium chloride (MitoTEMPO), N-acetyl-L-cysteine choline ester (MitoGSH), glutathione choline ester (MitoGSH), SS-02 tetrapepetide (Dmt-D-Arg-Phe, Lys-NH₂), SS-20 tetrapepetide (Phe-D-Arg-Phe-Lys-NH₂), SS-31 tetrapeptide (D-Arg-Dmt-Lys-Phe-NH₂), β-nicotinamide adenine dinucleotide, a pharmaceutically acceptable salt thereof, or any combination thereof.

6. The method of claim 1, wherein said subject is diagnosed as having suffered a myocardial infarction at least 24 hours prior to said administering step.

7. The method of claim 1, wherein the antioxidant comprises 2-(2,2,6,6-tetramethylpiperidin-1-oxyl-4-ylamino)-2-oxoethyl)triphenylphosphonium chloride (MitoTEMPO).

8. The method of claim 1, wherein a pharmaceutically effective amount of the antioxidant is administered to the subject to restore the sodium current (I_{Na}) to at least 80% of normal activity.

9. The method of claim 1, wherein the antioxidant restores said sodium current (I_{Na}) to at least 80% of normal activity by reducing or suppressing mitochondrial reactive oxygen species (ROS) production.

10. The method of claim 1, wherein the subject suffered from diabetes mellitus, pathological cardiac hypertrophy, myocardial ischemia/reperfusion, ischemic cardiomyopathy, heart failure, hypertension, atherosclerosis, valvular or coronary artery disease.

11. The method of claim 1, wherein the arrhythmia is selected from atrial and ventricular arrhythmias.

12. The method of claim 1, wherein the antioxidant is administered at a dose in the range of 0.01-5.0 mg/kg once or twice a day.

13. The method of claim 1, wherein the antioxidant if administered intravenously, subcutaneously, intraperitoneally, intravitreally, or orally.

14. The method of claim 1, wherein the subject is a human.

15. The method of claim 1, wherein said subject is diagnosed as having suffered a myocardial infarction at least 48 hours prior to said administering step.

16. A pharmaceutical composition comprising a compound of formula: 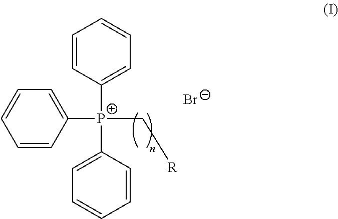
or a pharmaceutically acceptable salt thereof,
wherein
n is an integer from 0 to 15;
R is selected from a group consisting of CH₃, 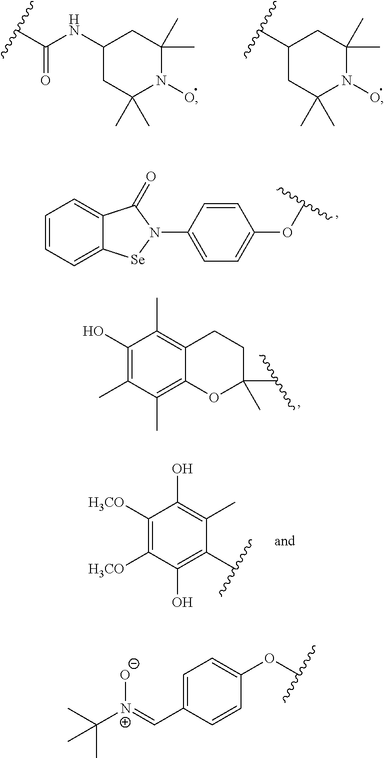
and a pharmaceutical acceptable carrier, diluent or excipient.

17. A pharmaceutical composition of claim 16 for use in suppressing or reducing mitochondrial reactive oxygen species (ROS) production in a cardiac cell.

18. A pharmaceutical composition of claim 16 for use in modulating or controlling sodium channel current of a cardiac cell.

19. The method of claim 1, wherein said mitochondrial-targeted antioxidant or the pharmaceutically acceptable salt thereof is administered at least 7 days after said subject has been diagnosed with having suffered a myocardial infarction.

20. The method of claim 1, wherein said mitochondrial-targeted antioxidant or the pharmaceutically acceptable salt thereof is administered 7 to 21 days after said subject has been diagnosed with having suffered a myocardial infarction.

21. The method of claim 1, wherein the mitochondrial-targeted antioxidant or the pharmaceutically acceptable salt thereof is administered before, during, or after surgical treatment of said acute arrhythmia of said subject.

22. The method of claim 18, wherein the mitochondrial-targeted antioxidant or the pharmaceutically acceptable salt thereof is administered 24 hours to 1 minute before the surgical treatment.

23. The method of claim 18, wherein the mitochondrial-targeted antioxidant or the pharmaceutically acceptable salt thereof is administered 1 minute to 2 days after the surgical treatment.

24. The method of claim 1, wherein the mitochondrial-targeted antioxidant or pharmaceutically acceptable salt thereof is present as a coating on surgical equipment comprising, a stent, an angioplasty balloon, drug-eluting suture, staple and tack, and administered to a patient undergoing cardiovascular surgery.

25. A method of inhibiting a VT-storm, comprising administering a mitochondrial-targeted antioxidant to a subject in need thereof.

26. The method of claim 25, wherein the VT storm comprises at least 3 sustained episodes of VT.

27. The method of claim 25, wherein the VT storm episode lasts for at least 30 seconds.

28. A cardiac surgical device comprising a mitochondrial-targeted antioxidant.

29. The device of claim 28, wherein said cardiac surgical device is a stent.

30. The device of claim 28, wherein said cardiac surgical device is an angioplasty balloon.

31. A mitochondrial-targeted antioxidant for use in reducing mitochondrial reactive oxygen species (ROS) production in a cardiac cell or modulating sodium channel current of a cardiac cell.
